(19) United States
(12) Patent Application Publication
(10) Pub. No.: US 2022/0380713 A1
(43) Pub. Date: Dec. 1, 2022

(54) AN IN VITRO ENDOTHELIAL CELL CULTURE SYSTEM FOR OPTIMIZING PULSATILE WORKING MODES OF THE CONTINUOUS FLOW ARTIFICIAL HEART

(71) Applicant: DALIAN UNIVERSITY OF TECHNOLOGY, Dalian, Liaoning (CN)

(72) Inventors: Yu WANG, Dalian, Liaoning (CN); Lixue LIANG, Dalian, Liaoning (CN); Kairong QIN, Dalian, Liaoning (CN); Yongjiang LI, Dalian, Liaoning (CN); Chundong XUE, Dalian, Liaoning (CN); Junwei WANG, Dalian, Liaoning (CN); Jingtong NA, Dalian, Liaoning (CN); Yunong YANG, Dalian, Liaoning (CN)

(21) Appl. No.: 17/617,887

(22) PCT Filed: Feb. 22, 2021
(86) PCT No.: PCT/CN2021/077163

§ 371 Date: Dec. 9, 2021

(30) Foreign Application Priority Data
Feb. 4, 2021 (CN) 202110166567.6

(51) Int. Cl. C12M 1/00; C12M 3/06; C12M 1/42; C12M 1/34; C12M 1/36

(52) U.S. Cl. CPC C12M 29/12; C12M 23/16; C12M 23/26; C12M 29/18; C12M 35/04; C12M 41/40; C12M 41/48; C12M 41/46

(57) ABSTRACT

An in vitro endothelial cell culture system for optimizing the pulsatile working mode of a continuous flow artificial heart belongs to the technical field of artificial organs. The system includes three parts: 1) a cell culture model on a microfluidic chip and an off-chip multielement aortic arch afterload fluid mechanics circulation loop; 2) devices for simulating the power source of a cardiovascular system: a fluid loading device is realized by a pulse blood pump, and an artificial heart device is connected in parallel to both ends of the pulse blood pump; and 3) a peripheral detection and feedback control system, comprising pressure and flow sensors, a fluorescence microscope, a CCD high-speed camera system and a proportional-integral-derivative feedback control system. The system can accurately simulate the real hemodynamics microenvironment of vascular endothelial cells in different parts of the aortic arch.

### TECHNICAL FIELD

The present invention belongs to the technical field of artificial organs, relates to an endothelial cell culture model and an in vitro circulatory system for optimizing the pulsatile working mode of an artificial heart, and is a miniature in vitro mock circulatory system for studying the influence of change in hemodynamics signals on the functions of endothelial cells caused by different pulsatile working modes of the pump speed of the continuous flow artificial heart based on the hemodynamics principle, the microfluidic chip technology and, the intelligent feedback control technology.

### BACKGROUND

At present, an artificial heart is a non-pharmacological mechanical treatment and rehabilitation method for end-stage heart failure. A continuous flow artificial heart is popularized and applied greatly due to its small size, high reliability, and easy implantation and operation. The main body is an impeller type blood pump, and the impeller generatessteady flow when rotating at a constant speed and outputs pulsatile flow when rotating at a speed changing periodically. In clinical use, the impeller pump is usually set at a constant speed for convenient operation. This working mode will lead to a significant decrease in pulsatility of arterial blood flow and blood pressure and cause dysfunction of vascular endothelial cells, thus inducing a lot of adverse events such as arteriovenous malformation, hemorrhagic stroke, and damage to the kidney and other organs.

Basic and clinical studies with more than 20 years show that artificial hearts can affect arterial endothelial functions through the hemodynamics mechanism, and further regulate the structure and functions of peripheral vessels. As a barrier between blood flow and vascular wall tissues, vascular endothelium is located at the innermost layer of the vascular wall, thus in a complex hemodynamics microenvironment. Meanwhile, vascular endothelium directly bears the wall shear stress and blood pressure caused by blood flow, and the effect of hemodynamics signals such as vascular circumferential stretch stress (or strain) caused by blood pressure. Endothelial cells can recognize hemodynamics signals and changes thereof in the extracellular microenvironment through cell membrane surface receptors and sensory receptors, and transfer the hemodynamics signals into the cells through a series of signaling pathway cascades to induce changes in gene and protein expression, i.e., mechanobiology mechanism, finally affecting changes in functions of endothelial cells, for example, secretion of vasoactive substances such as endothelium-derived relaxing factor, nitric oxide (NO) and endothelium-derived constricting factor, endothelin-1 (ET-1), and changes in expression levels of proinflammatory cytokines such as tumor necrosis factor-α (TNF-α), interleukin-6 (IL-6) and interleukin-8 (IL-8). The short-term effects of the substances will affect the relaxation and contraction functions of vascular walls and cause inflammatory responses, while the long-term effects will lead to the reconstruction of vascular structure and functions, i.e., changes in vascular wall thickness, vascular diameter, and vascular elasticity.

The current studies have set the blood pump speed of a continuous flow artificial heart to various pulsatile working modes with periodic changes, the amplitude regulation of the periodic pump speedis mainly set according to the hemodynamics mechanism of the coupling effect between ventricle and afterload, the frequency regulation comprises synchronous regulation (the frequency of pump speed change is consistent with the heart rate) and asynchronous regulation (the frequency of pump speed change is irrelevant to the heart rate), and it is expected to improve the vascular endothelial function and reduce the incidence rate of adverse events in peripheral vessels and organs by changing the pulsatility of hemodynamics signals. However, how different pulsatile working modes of the pump speed of the continuous flow artificial heart affect the law of the hemodynamics signals, and how to differently regulate the functions of arterial endothelial cells have not been comprehensively and systematically studied so far, thus limiting the accurate implementation in clinical practice and the scientific and reasonable use in treatment and rehabilitation of different pulsatile working modes of the artificial heart pump speed.

Animal models and human clinical experiments are the most direct methods to analyze the characteristics of arterial endothelial hemodynamics microenvironment before application of the continuous flow artificial heart in clinical practice. However, the hemodynamics microenvironment of in vivo arterial endothelial cells of animals and human bodies is very complex, is easily affected by other factors such as respiration and nervous regulation, and meanwhile, has the issuesof high individual difference, low accuracy, high cost and long cycle of hemodynamics parameter monitoring, and ethical controversy. Aiming at the above limitations, in the current studies, mechanical pumps have beenused to simulate ventricles, various distributed parameters (silicone elastic tube, etc.) or lumpedparameter elements (vascular compliance, blood flow inertia, peripheral resistance, etc.) have beenused to simulate the input impedance of arterial afterload, and the in vitro mock circulatory system (MCS) modelscoupled with theartificial heart have beenestablished. However, in these studies, the blood pressure, wall shear stress, and stretch strain in the local microenvironment near arterial endothelial cells have not been analyzed in details, and meanwhile, defects that the size of MCS models is large, the amount of circulating fluid is large and no in vitro cell culture model is contained generally exist, which is not convenient for cellular mechanobiologybased research. In comparison, the microfluidic chip has the advantages of a small quantity of samples required, easy integration, easy optical detection, and good biological adaptability. Recent studies show that the in vitro endothelial cell culture model (ECCM) based on the microfluidic chip is a miniaturized, objectified, standardized, and quantified mechanobiology research system for endothelial cells capable of simulating and easily monitoring hemodynamics microenvironment signals. However, the ECCM currently established to study the influence of the hemodynamics microenvironment corresponding to different pulsatile working modes of the artificial heart pump speed on the functions of arterial endothelial cells fails to reproduce real hemodynamics signals in the human arterial endothelial microenvironment under the action of an artificial heart. Therefore, it is urgent to design and construct a miniature in vitro mock circulatory system capable of accurately simulating the in vivo arterial endothelial hemodynamics microenvironment, which can not only realize the precise loading and control of hemodynamics signals under different pulsatile working modes of the artificial heart pump speed, but also realize real-time and online monitoring on the mechanobiology effects of arterial endothelial cells in the cell culture model on a microfluidic chip, so as to better analyze key hemodynamics signals such as blood pressure, wall shear stress and stretch stress (or strain) in the local microenvironment near arterial endothelial cells under different pulsatile working modes, thus providing scientific basis for the optimal selection of the pulsatile working mode of the artificial heart pump speed to improve the ability of theartificial hearts to treat and recover heart failure.

### SUMMARY

The purpose of the present invention is to provide a method capable of really simulating blood pressure, wall shear stress and stretch strain (stress) signals in the arterial endothelial hemodynamics microenvironment caused by pulsatile working modes of an artificial heart. The method ingeniously combines the hemodynamics principle, the microfluidic chip technology and the intelligent feedback control technology, constructs an in vitro fluid mock circulatory system by the cell culture model on a microfluidic chip with higher integration and lower material consumption and a multielement lumpedparameter model characterizing the afterload hemodynamics characteristics, and reproduces the combined effect of pressure, shear stress and stretch strain on in vivo arterial endothelial cells under different pulsatile working modes of the pump speed of a heart failure patient implanted with an artificial heart, which can be used to study the quantitative relationship between hemodynamics signals and the mechanobiology effects of arterial endothelial cells and the molecular biology mechanism thereof.

The present invention has the following technical solution:

An in vitro endothelial cell culture system for optimizing the pulsatile working mode of a continuous flow artificial heart (as shown in FIG. 1), containsthree basic units: the first basic unit is a cell culture model on a microfluidic chip and an off-chip multielement aortic arch afterload fluid mechanics circulation loop (as shown in FIG. 2), including a flow inductance, a resistance valve, an elastic chamber 1 and an elastic chamber 2 which are connected in series with the cell culture model, and the elastic chamber 1 and the elastic chamber 2 are arranged on both sides of the cell culture model;

The second basic unit is a pulse fluid loading device and an artificial heart device for simulating the power source of a cardiovascular system, as shown in FIG. 2, wherein the fluid loading device is realized by a pulse blood pump (Q₁(t) in FIG. 3) thatcan simulate the waveforms of blood pressure, wall shear stress and stretch strain on in vivo arterial endothelial cells of healthy persons and heart failure patients,and the artificial heart device is connected in parallel to both ends of the pulse blood pump (Q₂(t) in FIG. 3), and the pulse fluid loading device and the artificial heart device are connected in series to the fluid mechanics circulation loop, which can simulate signals of blood pressure, wall shear stress and stretch strain on in vivo arterial endothelial cells under different pulsatile modulation modes of the pump speed;

The third basic unit is a peripheral detection and feedback control system, as shown in FIG. 1, comprising an inverted fluorescence microscope, a CCD high-speed camera system, a pressure sensor, a flow sensor and a proportional-integral-derivative (PID) feedback control system, wherein the pressure sensor and the flow sensor are arranged on both sides of the cell culture model for real-time monitoring and acquisition of pressure and flow waveforms of the input end and the output end of the cell culture model, the fluorescence microscope is located above the cell culture model Rc, the CCD high-speed camera system is connected with the fluorescence microscope for acquiring the actual morphological structure of cells in the cell culture model on a microfluidic chip, the CCD high-speed camera system, the pressure sensor and the flow sensor are all connected with the PIDfeedback control system, and by acquisition and feedback of the pressure and flow waveforms of both ends of the cell culture model and the morphological structure data of cells, the PID feedback control system can quantitatively regulate changes in related hemodynamics signals and produce the combined effect of signals of pressure, shear stress and stretch strain on in vivo arterial endothelial cells under different pulsatile working modes of the artificial heart pump speed in the cell culture model on a microfluidic chip.

As shown in FIG. 4, the above endothelial cell culture model is a cavity with a concave section, an elastic film with the elastic modulus similar to that of an artery is bonded to a cavity made of hard and transparent polymethyl methacrylate (PMMA), and the cell culture model below the lower surface of the elastic film is full of circulating fluid; air is introduced into cavities on both sides of the upper surface of the elastic film to provide enough space for the deformation of the film at the cavities on both sides under the action of pulsating fluid pressure; both ends of a cavity in the middle of the upper surface of the elastic film are smooth and cambered, the middle part of the upper surface of the film is close to the inner surface of the concave part of the cavity in the horizontal direction, which enables the lower elastic film adhering to endothelial cells to produce horizontal stretch strain under the action of stretch of both sides, and the concave thickness of the cavity shall be designed to ensure that the microscope can focus when being used for observing the morphological structure of endothelial cells and the cavity will not deform under the action of pulsating pressure; and the geometric size of the lower cell culture model and the elastic modulus of the elastic film shall be selected according to the principle of elastic mechanics, and determined by accurately simulating the actual need of the waveforms of blood pressure, shear stress and stretch strain in the endothelial microenvironment of different parts of aorta.

Further, the cell culture model provides circulating fluid for cells in the cell culture model through matching of the resistancevalve and the reservoir.

Further, the in vitro circulatory system can be equivalent to a circuit model, wherein the flow resistance of the endothelial cell culture model is equivalent to a resistor (Rc in FIG. 3), the compliance of the film on the culture model is equivalent to a capacitor (C₁ in FIG. 3), and the compliance, the flow resistance, and the flow inductance of an aortic arch downstream vascular bed are equivalent to a capacitor, a resistor, and an inductor (C₂, R, and L in FIG. 3), respectively.

Further, the off-chip multielement aortic arch afterload fluid mechanics circulation loop shall be designed to keep the pressure, wall shear stress, and stretch strain on endothelial cells cultured on the film of the cell culture model consistent with the waveforms of blood pressure, shear stress, and stretch strain on endothelial cells of the corresponding part of a heart failure patient implanted with an artificial heart:

First, with the waveforms of blood pressure p(t), wall shear stress τ_{ω} (t) and stretch strain ε(t) near local in vivo arterial endothelial cells obtained from the detection and analysis of human or animal experiments as a simulated target, to make the waveforms of blood pressure and shear stress on the endothelial cells cultured on the film of the cell culture model equal to blood pressure and wall shear stress in the in vivo arterial endothelial microenvironment, blood flow q(t) and pressure drop Δp(t) must satisfy:

$$q ( t ) = \frac{{W}_{c} {H}_{c}^{2}}{6 η} {τ}_{ω} ( t ) \qquad ( 1 a )$$

$$max ( \frac{Δ p ( t )}{p ( t )} ) = max ( \frac{2 {L}_{c} {τ}_{ω} ( t )}{{H}_{c} p ( t )} ) << 1 \qquad ( 1 b )$$

wherein η is the viscosity of the cell culture fluid, and Hc, Wc and Lc are respectively height, width and length of the cell culture model.

Second, the hemodynamics behavior of the aortic arch afterload is equivalent to a circuit system according to the similarity relationship between the fluid mechanics loop and the circuit,wherein the input impedance of the fluid mechanics loop is expressed as the ratio of the input pressure waveform p(t) to the blood flow waveform q(t) in the frequency domain and characterized by the amplitude and the phase of harmonic components of blood pressure and blood flow corresponding to the angular frequency con:

$$\vert z ( {ω}_{n} ) \vert = \frac{\vert P ( {ω}_{n} ) \vert}{\vert Q ( {ω}_{n} ) \vert} \qquad ( 2 a )$$

$$∠ z ( {ω}_{n} ) = ∠ P ( {ω}_{n} ) - ∠ Q ( {ω}_{n} ) \qquad ( 2 b )$$

wherein |P(ωₙ)| and |Q(ωₙ)| are respectively amplitudes of blood pressure and blood flow at the angular frequency ωₙ after Fourier transform; [symbol] P(ωₙ) and [symbol] Q(ωₙ) are respectively phase angles of blood pressure and blood flow at the angular frequency ωₙ after Fourier transform; and |z(ωₙ)| and [symbol] z(ωₙ) are respectively the amplitude and the phase angle of the input impedance of the aortic arch downstream afterload at ωₙ. The input impedance of the equivalent circuit model is expressed as a complex function composed of circuit elements in FIG. 3, and the parameter value of each element of an equivalent lumpedparameter circuit model is determined by the system identification method in formula 3 based on the amplitude-frequency curve and the phase-frequency curve of the input impedance in the fluid mechanics loop.

$$R S S = {\sum }_{{i = 1}^{}}^{N} \frac{{( \vert z ( {ω}_{i} ) \vert - \vert \hat{z} ( {ω}_{i} ) \vert )}^{2}}{{\vert z ( {ω}_{i} ) \vert}^{2}} + {\sum }_{{i = 1}^{}}^{N} \frac{{( ∠ z ( {ω}_{i} ) - ∠ \hat{z} ( {ω}_{i} ) )}^{2}}{p {i}^{2}} \qquad ( 3 )$$

wherein |\hat{z}(ωᵢ)| and <\hat{z}(ωᵢ) are respectively the amplitude and the phase angle of the equivalent input impedance of the lumpedparameter circuit model ωₙ.

Finally, a multielement in vitro fluid mock circulatory system for simulating the hemodynamics characteristics of the aortic arch afterload is built according to the numerical values of the flowinductanceL, the resistance valve R, the elastic chamber C₁ and the elastic chamber C₂.

As shown in FIG. 1 and FIG. 2, the circulating fluid in the system is the endothelial cell culture solution of in vitro blood vessels, the elastic chambers simulate arterial compliance (fluid capacitance), the resistance valve simulates viscous resistance (flow resistance), and the flow inertiaelement simulates flow inertia (inductance). It is worth pointing out that due to different waveforms of blood pressure, shear stress and stretch strain near the walls of different parts of the aortic arch, the equivalent circuit for describing the hemodynamics behavior of the afterload system in the different parts of the aortic arch may not be unique and needs to be adjusted according to the actual waveforms. The desired blood flow waveform is generated by the pulse blood pump and the artificial heart. Once the input blood flow waveform of the cell culture model is known, the pressure waveform is uniquely determined according to the above equivalent circuit.

The above device for simulating the power source of a cardiovascular system is realized by an artificial heart connected in parallel to both ends of the pulse fluid loading device. The pulse fluid loading device can be used in combination with the PID feedback control device to simulate signals of blood pressure, wall shear stress and stretch strain in the hemodynamics microenvironment of in vivo arterial endothelial cells under normal and heart failure physiological conditions, the artificial heart device and the fluid loading device are connected in parallel and then connected in series to the above fluid mechanics circulation loop and can produce hemodynamics signal waveforms borne by vascular endothelial cells of different parts of the aortic arch under different pulsatile working modes of the artificial heart pump speed in combination with the PID feedback control device, the acquired signals are fed back to the PID control device to further regulate the fluid loading device and the artificial heart, so as to quantitatively regulate changes in the amplitude and frequency of pressure and flow signals on the multielement mock circulatory system,and finally produce the combined effect of blood pressure, shear stress and stretch strain under different pulsatile working modes of the artificial heart pump speed in the cell culture model on a microfluidic chip.

The experimental steps for using the above system to study the quantitative relationship between different pulsatile working modes of the pump speed of the continuous flow artificial heart and hemodynamics signals of the local arterial endothelial microenvironment are as follows:

Step 1: carrying out subculturing on primarily cultured endothelial cells, wherein the 2^{nd} to 5^{th} generations are used for experiments. Adjusting the numerical values of the flowinductanceL, the resistance valve R, the elastic chamber C₁ and the elastic chamber C₂ in the in vitro mock circulatory system, introducing circulating fluid into the cell culture model, and loading combined stimulation of various hemodynamics signals of arterial endothelial cells under different pulsatile working modes of the pump speed of the continuous flow artificial heart by regulating the pulse blood pump and the artificial heart device.

Step 2: continuing loading hemodynamics signal stimulation corresponding to the above working mode, and then carrying out activity detection on the cells to ensure the effectiveness of the above system.

Step 3: collecting cell samples from the cell culture model on a microfluidic chip, and determining gene and protein expression levels, so as to analyze the influence of hemodynamics signals such as blood pressure, shear stress and stretch strain caused by different pulsatile working modes of the artificial heart pump speed on the gene and protein expression levels of vasoactive substances and proinflammatory cytokines.

The present invention has the following beneficial effects: the present invention can successfully reproduce signals of blood pressure, wall shear stress and stretch strain corresponding to different pulsatile working modes of the artificial heart pump speed based on the above in vitro mock circulatory system, use a cell culture model on a microfluidic chip with higher integration level and less material consumption to study the differentiated influence of the combined stimulation of hemodynamics signals on the functions of arterial endothelial cells in the above working modes,provide an efficient and reasonable experimental platform for studying the quantitative relationship between hemodynamics signals and the functions of arterial endothelial cells, and provide a scientific basis for selecting the pump speed working mode of a continuous flow artificial heart which is more beneficial to improvement and maintenance of normal endothelial functions.

### DESCRIPTION OF DRAWINGS

FIG. 1 is a structural schematic diagram of an in vitro endothelial cell culture model and a periphery monitoring system.

FIG. 2 is a schematic diagram of a fluid mechanics circulation loop of an in vitro endothelial cell culture model.

FIG. 3 is a schematic diagram of an equivalent circuit model of hemodynamics behaviors of aortic arch afterload.

FIG. 4 is a schematic diagram of a cell culture model on a microfluidic stretch chip.

FIG. 5 is a schematic diagram showing the waveforms of blood pressure and shear stress on vascular endothelial cells of the aortic arch obtained through in vivo experiments and the waveform of blood flow in the culture model obtained by inverse solution according to the waveform of shear stress and the size of the cell culture model in normal, heart failure and asynchronous modulation modes.

FIG. 6 is a schematic diagram showing the results of fitting the amplitude and the phase angle of the actual input impedance by Matlab/Simulink in normal, heart failure and asynchronous modulation modes of the above equivalent circuit model and the results of comparing the output voltage of the model as the simulation target with the blood pressure in FIG. 5 with the blood flow information as current excitation; (a-1): an amplitude-frequency curve of the input impedance under normal physiological conditions; (a-2): a phase angle-frequency curve of the input impedance under normal physiological conditions; (a-3): a comparison diagram of the output voltage of the model and the blood pressure under normal physiological conditions; (b-1): an amplitude-frequency curve of the input impedance under heart failure conditions; (b-2): a phase angle-frequency curve of the input impedance under heart failure conditions; (b-3): a comparison diagram of the output voltage of the model and the blood pressure under heart failure conditions; (c-1): an amplitude-frequency curve of the input impedance under the asynchronous pulsatile working mode of the artificial heart pump speed; (c-2): a phase angle-frequency curve of the input impedance under the asynchronous pulsatile working mode of the artificial heart pump speed; and (c-3): a comparison diagram of the output voltage of the model and the blood pressure under the asynchronous pulsatile working mode of the artificial heart pump speed.

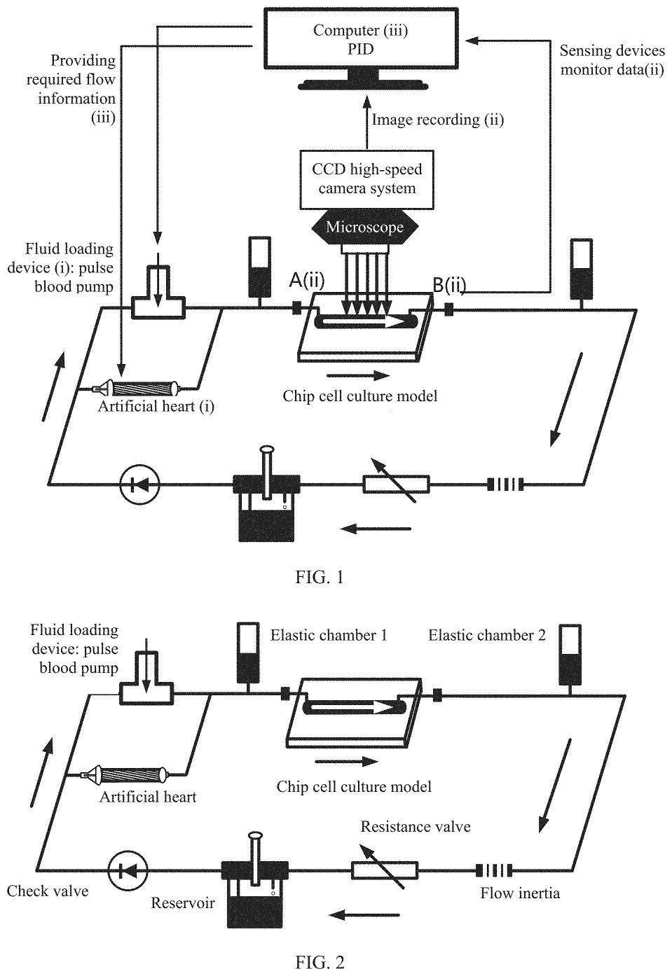

FIG. 1 includes a fluid loading device—a pulse blood pump (i) and an artificial heart (i); a signal acquisition and processing system (ii) comprising an inverted fluorescence microscope, a CCD high-speed camera system, pressure and flow sensors, and a proportional-integral-derivative (PID) feedback control system (iii); A(ii) and B(ii) are pressure and flow sensors located on both ends of the cell culture model on a microfluidic chip; Rc is the flow resistance of a cell culture model on a microfluidic chip; C₁ is the compliance of a film; R is the flow resistance of the connecting tube; C₂ is an elastic air chamber characterizing compliance; and L is the flow inductance of the connecting tube in the fluid circulation process.

### DETAILED DESCRIPTION

The specific implementation solution of simulating blood pressure in the arterial endothelial hemodynamics microenvironment under different pulsatile working modes of the artificial heart pump speed is described as follows:

(1) The height Hc, the width We and the length Lc of the cell culture model on a microfluidic chip are respectively designed to be 0.3 mm, 6 mm and 15 mm, and the viscosity η of the cell culture fluid is usually 0.001 Pa·s; and three target input impedances z(ωᵢ) are respectively calculated according to the target blood pressure and blood flow in three physiological conditionsin FIG. 5;

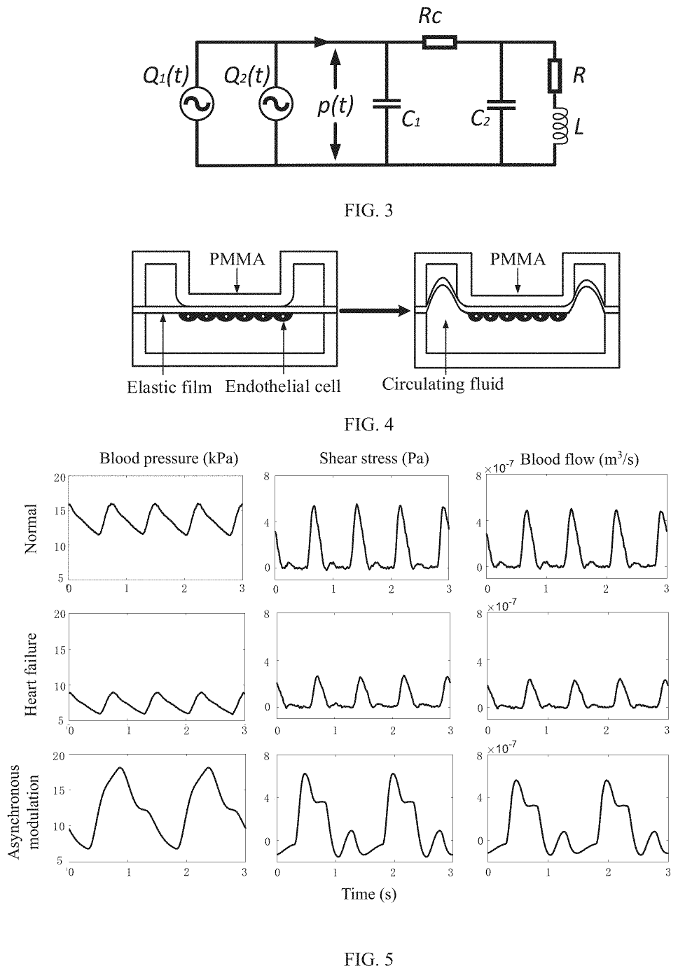

(2) The hemodynamics characteristics of the in vitro mock circulatory system can be characterized by the five-element equivalent circuit model shown in FIG. 3, and it can be known from the related circuit theory that the input impedance \hat{z}(ωᵢ) of the circuit can be expressed as follows:

$$\hat{z} ( {ω}_{i} ) = \frac{{R}_{c} L {{C}_{2} ( j {ω}_{i} )}^{2} + ( {R}_{c} R {C}_{2} + L ) ( j {ω}_{i} ) + {R}_{1} + {R}_{2}}{\begin{matrix}{R}_{c} {C}_{1} {C}_{2} {L ( j {ω}_{i} )}^{3} + ( {R}_{c} R {C}_{1} {C}_{2} + {C}_{1} L + {C}_{2} L ) {( j {ω}_{i} )}^{2} + \\ ( {R}_{c} {C}_{1} + R {C}_{1} + R {C}_{2} ) ( j {ω}_{i} ) + 1\end{matrix}} \qquad ( 4 )$$

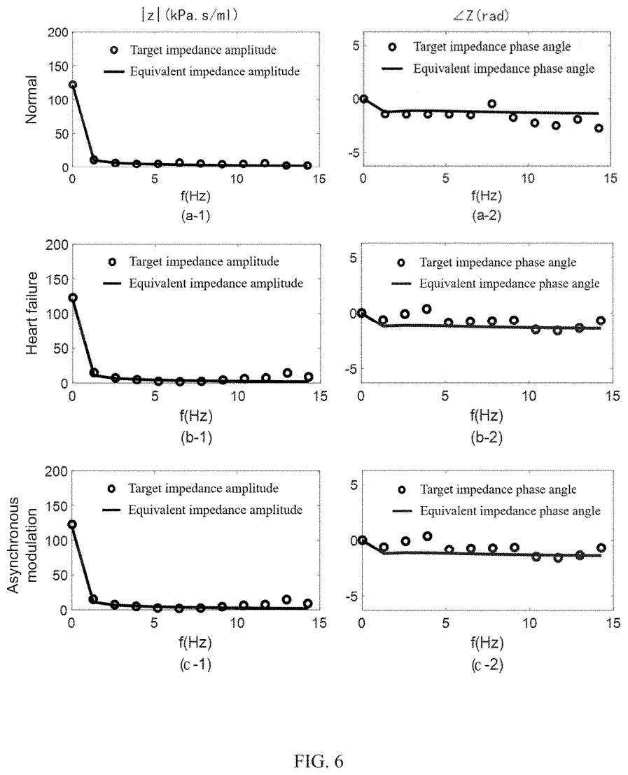

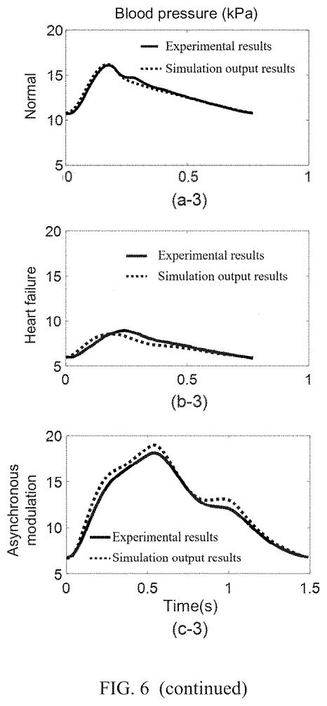

(3) Formula 4 shows the equivalent input impedance \hat{z}(ωᵢ) of the five-element lumpedparameter model, and the parameter value of each element can be obtained through the system identification method in combination with thetarget input impedance z(ωᵢ), wherein the parameter value of each element in the corresponding fluid mechanics loop in the normal physiological status is respectively Rc=8.6 kPa·s/ml, R=113.06 kPa·s/ml, C₁=0.0053 ml/kPa, C₂=0.0097ml/kPa and L=19.2972 kPa·s²/ml,and the parameter value of each element in the corresponding fluid mechanics loop in the heart failure status and under the asynchronous pulsatile working mode of the pump speed after implantation of an artificial heart is respectively Rc=13 kPa·s/ml, R=109 kPa·s/ml, C₁=0.005 ml/kPa, C₂=0.009 ml/kPa and L=1 kPa·s²/ml. As shown in FIG. 6, the input impedance curve (solid line in FIG. 6) corresponding to the five-element lumpedparameter model and the target input impedance curve (circle in FIG. 6) basically coincide. Based on the parameter values of the elements in the above three different physiological conditions, after the corresponding input blood flow waveform is given, the blood pressure waveform obtained by Matlab/simulink simulation is basically consistent with the corresponding blood pressure waveform in FIG. 5, as shown in FIG. 6, and the root mean square errors are 0.237, 0.401 and 0.625 respectively; and then the fluid mechanics circulation loop based on the cell culture model on a microfluidic chip as shown in FIG. 2 is constructed;

(4) The chip is made through a standardized micromachining method, an elastic film with the elastic modulus similar to that of an artery is bonded to a cavity which is made of hard and transparent PMMA and has a concave section, and the cell culture model below the lower surface of the elastic film is full of circulating fluid; air is introduced into cavities on both sides of the upper surface of the elastic film to provide enough space for the deformation of the film at the cavities on both sides under the action of pulsating fluid pressure; both ends of a cavity in the middle of the upper surface of the elastic film are smooth and cambered, the middle part of the upper surface of the film is close to the inner surface of the concave part of the cavity in the horizontal direction, which enables the lower elastic film adhering to endothelial cells to produce horizontal stretch strain under the action of stretch of both sides, and the concave thickness of the cavity shall be designed to ensure that the microscope can focus when being used for observing the morphological structure of endothelial cells and the cavity will not deform under the action of pulsating pressure; and the geometric size of the lower cell culture model and the elastic modulus of the elastic film shall be selected according to the principle of elastic mechanics, and determined by accurately simulating the actual need of the waveform of blood pressure, shear stress and stretch strain in the endothelial microenvironment of different parts of aorta;

(5) An in vitro endothelial cell culture model and a periphery monitoring system shown in FIG. 1 is established, includinga pulse blood pump (i), an artificial heart (i), a signal acquisition and processing system (ii) composed of a plurality of components, and a PID feedback control system (iii). The signal acquisition and processing system (ii) comprises an inverted fluorescence microscope, a CCD high-speed camera system, pressure and flow sensors, and is used for real-time monitoring and acquisition of pressure and flow waveforms of the input end A and the output end B of the cell culture model, and the actual morphological structure of cells in the cell culture model on a microfluidic chip. The pulse blood pump (i) can accurately simulate normal and heart failure physiological conditions in combination with the PID feedback control device (iii), and the artificial heart device (i) is connected in parallel to both ends of the pulse blood pump, and can accurately simulate signals of blood pressure, wall shear stress and stretch strain on vascular endothelial cells of specific parts of the aortic arch under different pulsatile working modes of the artificial heart pump speed in combination with the PID feedback control device (iii), to finally load quantitative and controllable pulsating flow signals into the multielement mock circulatory system; The acquired signals are fed back to the PID control device (iii) to further regulate the pulse blood pump (i) and the artificial heart (i), so as to quantitatively regulate changes in the amplitude and frequency of pressure and flow signals on the multielement mock circulatory system, and finally produce the combined effect of blood pressure, shear stress and stretch strain under different pulsatile working modes of the artificial heart pump speed in the cell culture model on a microfluidic chip.

The pressure in the microfluidic chip can be measured by the pressure sensor; the shear stress can be calculated according to the flow measured by the flow sensor and the geometric size of the cell culture model; and the horizontal stretch strain of the elastic film in the chip under different pressures can be calibrated on the film by using fluorescent microspheres and measured by the fluorescence microscope. The strain corresponding to the elastic film is obtained by giving different pressures, so as to establish a relational expression between the pressures and the stretch strain. According to the approximate expression, the stretch strain of the elastic film in the actual experiment is determined under the condition that the pressure is known. In addition, the morphological structure of endothelial cells is detected and recorded by the microscope in combination with the CCD high-speed camera system and saved to the computer.

(6) The specific experimental steps for studying the quantitative relationship between different pulsatile working modes of the pump speed of the continuous flow artificial heart and hemodynamics signals of a local arterial endothelial microenvironment are as follows:
Step 1: carrying out subculturing on primarily cultured endothelial cells by an EGM culture medium, wherein the 2^{nd} to 5^{th} generations are used for experiments. During experiments, endothelial cells are planted on an elastic film of a cell culture model on a microfluidic chip coated with Fibronection so that the cells adhere to the wall and the degree of fusion is more than 90%.
Step 2: loading combined stimulation of hemodynamics signals corresponding to different pulsatile working modes of the artificial heart pump speed on arterial endothelial cells; and using NucView™-488 cell activity detection reagent for cell activity detection to ensure the effectiveness of the in vitro mock circulatory system.
Step 3: collecting cell samples from the cell culture model on a microfluidic chip to determine gene and protein expression levels, so as to obtain the influence of hemodynamics signals such as blood pressure, shear stress and stretch strain corresponding to different pulsatile working modes of the artificial heart pump speed on the gene and protein expression levels of vasoactive substances and proinflammatory cytokines.

The present invention can successfully reproduce signals of blood pressure, wall shear stress and stretch strain on in vivo arterial endothelial cells in different pulsatile working modes of the artificial heart pump speed, and monitor the differentiated influence of the functions of arterial endothelial cells cultured under the combined stimulation of the above hemodynamics signals in real time.

1. An in vitro endothelial cell culture system for optimizing the pulsatile working mode of a continuous flow artificial heart, wherein the in vitro endothelial cell culture system comprises three basic units:
the first basic unit is a cell culture model on a microfluidic chip and an off-chip multielement aortic arch afterload fluid mechanics circulation loop; wherein the off-chip multielement aortic arch afterload fluid mechanics circulation loop comprises a flow inductance, a resistance valve, a first elastic chamber and a second elastic chamber which are connected in series with the cell culture model,and the first elastic chamber and the second elastic chamber are arranged on both sides of the cell culture model;
the second basic unit is a pulse fluid loading device and an artificial heart device for simulating the power source of a cardiovascular system, wherein the fluid loading device is realized by a pulse blood pump,and the artificial heart device is connected in parallel to both ends of the pulse blood pump, and then the pulse fluid loading device and the artificial heart device are connected in series to the off-chip multielement aortic arch afterload fluid mechanics circulation loop;
the third basic unit is a peripheral detection and feedback control system, includingan inverted fluorescence microscope, a CCD high-speed camera system, pressure andflow sensors, and a proportional-integral-derivative (PID) feedback control system, wherein the pressure and flow sensors are arranged on both sides of the cell culture model, the fluorescence microscope is located above the cell culture model Rc, the CCD high-speed camera system is connected with the fluorescence microscope, and the CCD high-speed camera system, the pressure and flow sensors are all connected with the PID feedback control system.

2. The in vitro endothelial cell culture system for optimizing the pulsatile working mode of a continuous flow artificial heart according to claim 1, wherein the cell culture model is a cavity with a concave section, an elastic film with the elastic modulus similar to that of an artery is bonded to a cavity, and the cell culture model below the lower surface of the elastic film is full of circulating fluid; air is introduced into cavities on both sides of the upper surface of the elastic film; the middle part of the upper surface of the elastic film is close to the inner surface of the concave part of the cavity in the horizontal direction; and both ends of the middle part of the upper surface of the elastic film are smooth and cambered.

3. The in vitro endothelial cell culture system for optimizing the pulsatile working mode of a continuous flow artificial heart according to claim 1, wherein the in vitro circulatory system is equivalent to a circuit model: the flow resistance of the endothelial cell culture model is equivalent to a resistor, the compliance of the film on the culture model is equivalent to a capacitor, and the compliance, the flow resistance and the flow inductance of an aortic arch downstream vascular bed are equivalent to a capacitor, a resistor and an inductor.

4. The in vitro endothelial cell culture system for optimizing the pulsatile working mode of a continuous flow artificial heart according to claim 1, wherein the off-chip multielement aortic arch afterload fluid mechanics circulation loop shall be designed to keep the pressure, wall shear stress and stretch strain on endothelial cells cultured on the film of the cell culture model consistent with the waveforms of blood pressure, shear stress and stretch strain on endothelial cells of the corresponding part of a heart failure patient implanted with an artificial heart:
first, with the waveform of blood pressure p(t), wall shear stress τ_{ω} (t) and stretch strain ε(t) near local in vivo arterial endothelial cells obtained from the detection and analysis of human or animal experiments as a simulated target, to make the waveforms of blood pressure and shear stress on the endothelial cells cultured on the film of the cell culture model equal to blood pressure and wall shear stress in the in vivo arterial endothelial microenvironment, blood flow q(t) and pressure drop Δp(t) must satisfy:
$$q ( t ) = \frac{{W}_{c} {H}_{c}^{2}}{6 η} {τ}_{ω} ( t ) \qquad ( 1 a )$$
$$max ( \frac{Δ p ( t )}{p ( t )} ) = max ( \frac{2 {L}_{c} {τ}_{ω} ( t )}{{H}_{c} p ( t )} ) << 1 \qquad ( 1 b )$$
wherein η is the viscosity of the cell culture fluid, and Hc, We and Lc are respectively height, width and length of the cell culture model;
second, the hemodynamics behavior of the aortic arch afterload is equivalent to a circuit model according to the similarity relationship between the fluid mechanics loop and the circuit, and the circuit model connects the flow inductorL characterizing the hemodynamics characteristics of the aortic arch downstream vascular bed with the flow resistor R in series, with the second elastic chamber C₂ in parallel, then with the flow resistance Rc of the cell culture model in series, and finally with the compliance C₁ of the film on the culture model in parallel, and the parameter values of the above elements in the lumpedparameter circuit model are determined by the system identification method;
finally, a multielement in vitro fluid mock circulatory system for simulating the hemodynamics characteristics of the aortic arch afterload is built according to the numerical values of the flow inductanceL, the resistance valve R, the first elastic chamber C₁ and the second elastic chamber C₂.

5. The in vitro endothelial cell culture system for optimizing the pulsatile working mode of a continuous flow artificial heart according to claim 1, wherein the cell culture model provides circulating fluid for cells in the cell culture model through matching of the resistance valve and the reservoir.

6. The in vitro endothelial cell culture system for optimizing the pulsatile working mode of a continuous flow artificial heart according to claim 2, wherein the pulse fluid loading device can be used in combination with the PID feedback control device to simulate signals of blood pressure, wall shear stress and stretch strain in the hemodynamics microenvironment of in vivo arterial endothelial cells under normal and heart failure physiological conditions, the artificial heart device and the fluid loading device are connected in parallel and then connected in series to the above fluid mechanics circulation loop, and can produce hemodynamics signal waveforms of different parts of the aortic arch under different pulsatile working modes of the artificial heart pump speed in combination with the PID feedback control device; and the acquired signals are fed back to the PID control device to further regulate the fluid loading device and the artificial heart, so as to quantitatively regulate changes in the amplitude and frequency of pressure and flowsignals on the multielement mock circulatory system, and finally produce the combined effect of blood pressure, shear stress and stretch strain under different pulsatile working modes of the artificial heart pump speed in the cell culture model on a microfluidic chip.
